(19) United States
(12) Patent Application Publication
(10) Pub. No.: US 2010/0318033 A1
(43) Pub. Date: Dec. 16, 2010

(54) INDWELLING TROCAR FOR POST-SURGICAL OPERATIONS

(76) Inventor: Albert Y. Lam, Palos Verdes Estates, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/483,656

(22) Filed: Jun. 12, 2009

(51) Int. Cl. A61B 17/34

(52) U.S. Cl. 604/164.11

(57) ABSTRACT

Methods and devices are provided that assist in reducing the risk of unintentional injury in patients during and after surgical procedures, including laparoscopic and open. Improved trocars are provided that include a cannula and an obturator. The cannula comprises first and second sections, the first section including the proximal end of the cannula closest to the surgeon and the second section including the distal end of the cannula proximate a surgical site. The second section of the cannula is of a material that is softer and/or more flexible than that of the first section. By being composed of a softer and/or more flexible material, the second section of the cannula can be maintained adjacent to or abut internal organs during and after a surgical procedure. In some cases, the improved trocar can have a cannula comprised of a single material such as rubber or silicone. The improved trocar can be safely maintained in a patient for a certain duration following a surgical procedure, such as after deflating the abdominal cavity in a laparoscopic procedure or after closing an incision in an open surgery. In some cases, the trocar cannula can be safely maintained in a patient up to seven days after completing a surgical procedure.

### BACKGROUND OF THE INVENTION

1. Field of the Invention

The present application relates to surgical instruments known as trocars, which are used to puncture anatomical cavities to provide communication with the cavities during surgical procedures. In particular, the present application relates to a trocar that can be maintained in a patient for a specified duration following deflation of the anatomical cavity or closure of an incision after a surgical procedure.

2. Description of the Related Art

Laparoscopic surgery, also referred to as minimally invasive surgery, is a type of surgery in which operations in the abdomen are performed through small incisions. Laparoscopic surgery provides many benefits to patients over open surgery techniques, including decreased post-operative recovery time, decreased post-operative pain and enhanced cosmetic results.

Trocars are sharp pointed surgical instruments used to puncture body cavities in laparoscopic surgeries. Trocars generally include a tube or cannula and piercing element referred to as an obturator. Once a body cavity is punctured by an obturator which slideably extends through and beyond the distal end of the cannula, the cannula can be inserted percutaneously into the body cavity to provide a portal through which drainage or surgical techniques can be performed.

In most instances, a laparoscopic surgical procedure begins with the inflation or insufflation of the abdominal cavity with a non-toxic gas, typically carbon dioxide. The introduction of the gas into the abdominal cavity lifts the abdominal wall away from the internal viscera. The abdominal wall is then pierced or penetrated using an obturator of a trocar that is slidably fit within the bore of the trocar cannula. After insertion of the cannula through the abdominal wall of the patient, the obturator is slidably removed while leaving the cannula protruding through the abdomen wall. Laparoscopic instruments can then be inserted through the cannula to view internal organs and to perform surgical procedures. After completion of the surgical procedures, the cannula is removed from the patient and the patient's body is then deflated and closed. Additional trocars may then be inserted under direct vision or the operation may be converted to an open procedure.

While laparoscopic surgery results in a reduced number of post-operative complications compared to open surgery procedures, post-operative problems still exist. Such post-operative complications include intra-abdominal infections, wound infections, bleeding, anastomotic leakage, mesenteric ischemia, mesenteric infarction, internal herniation, intestinal obstruction, kinking, twisting, narrow lumen, inadequate drainage of abdominal collections: bile and pancreatic secretions, enteric contents, etc. To detect such complications, a “second look” would be extremely useful. Generally, for patients who have post-operative complications or for which there is a risk of post-operative complications, the patient's abdominal cavity will be re-incised and re-insufflated. A surgeon will then use a trocar obturator to pierce through the abdominal wall of the patient, and once the obturator is removed, a laparoscope or camera can be inserted through the trocar cannula to scan for post-operative complications. A surgeon may, for example, examine the ischemic bowel or abdominal organs of a patient to seek out and correct any potential post-operative problems. Other equipment can also be delivered down the trocar cannula to perform specific post-operative procedures, including 5 mm cameras.

There are a number of problems associated with reinserting a trocar post-operation. Most importantly, reinserting a trocar post-operation can be hazardous to internal organs which abut the abdominal wall. Also, reinserting a trocar post-operation can be time-consuming, which is problematic when there is an urgent need to address a post-operative complication. Following surgical procedures, internal organs often adhere to the anterior abdominal wall because of fibrinous exudates, platelet aggregates, transundation of intravascular fluids. Intestines are generally distended post-operatively due to ileus and the abdomen may be tight. Reinsertion of an inflating needle could therefore puncture the bowel or other internal organs and is risky.

Despite the benefits of laparoscopic surgery, some patients may still undergo open surgeries, depending on the type of surgical procedure to be performed. In an originally open surgery or operation, a reopening of the original incision may be needed to allow for a “second look” for possible post-operative complications, which also results in risk of injury to a patient. The reluctance to reopen an abdomen for a “second look” can often delay the need to correct an abdominal complication and may lead to a missed opportunity that may result in added morbidity at best and death at worst for the patient.

Accordingly, there is a need for improved devices and methods for handling post-operative complications in laparoscopic and open surgeries.

### SUMMARY OF THE INVENTION

It is an object of the present application to provide improved devices and methods for handling post-operative complications in laparoscopic and open surgeries. The devices and methods described help facilitate the timely discovery of an accidental injury that occurred during surgery or progressive disease in patients with minimal risk of injury.

In accordance with some embodiments, a method for performing a surgical procedure on a subject is provided comprising inflating an abdominal cavity. After inflating the abdominal cavity, a trocar is inserted percutaneously to define an access path through the abdominal cavity. Several additional trocars may be inserted. One or more instruments can be provided through the trocar to assist in performing a surgical procedure. Following completion of the surgical procedure, the abdominal cavity can be deflated. The atraumatic trocar, which maybe used as an access path during the surgical procedure, can be maintained in the subject for a desired period of time following deflation of the abdominal cavity.

In accordance with some embodiments, a method for performing a surgical procedure in a body cavity of a patient is provided. A surgical procedure is performed in part through a trocar cannula extending percutaneously through the patient's skin toward a surgical site. After completion of the surgical procedure, the patient's body cavity is deflated, and the trocar cannula is maintained percutaneously in the body cavity for a desired period of time. The trocar cannula is designed to inhibit injury to tissue in the body cavity after deflation of the body cavity.

In accordance with some embodiments, a method for performing an open surgical procedure in a body cavity of a patient is provided. An incision is created in a patient and through the incision, a surgical procedure is performed. A trocar cannula is then inserted percutaneously into a body cavity of the patient, preferably through a separate incision away from the original incision. Following completion of the surgical procedure, the incision is closed partially or completely while maintaining the trocar cannula in place. Following closure of the incision, the trocar cannula can be kept in place to serve as an access port (e.g., for cameras) for a certain period of time.

In some embodiments, a trocar is provided comprising a cannula having a first section, a second section and an axial bore. The second section of the trocar is comprised of a more flexible material than the first section. The first section transitions into the second section, forming a continuous body around the axial bore. The trocar can also include a cannula obturator for piercing the abdominal wall of a patient for insertion purposes.

### BRIEF DESCRIPTION OF THE DRAWINGS

FIG. 1 illustrates a side view of a trocar according to some embodiments of the present application.

FIG. 2 is a flow chart of a method of laparoscopic surgery using a trocar assembly according to some embodiments of the present application.

FIG. 3 illustrates a cross-sectional view of a trocar in a body cavity according to some embodiments of the present application.

FIG. 4A illustrates a cross-sectional view of a trocar with an obturator in a body cavity according to some embodiments of the present application.

FIG. 4B illustrates a cross-sectional view of the trocar of FIG. 4A in a body cavity with the obturator removed.

FIG. 5 illustrates a cross-sectional view of an alternate trocar having a sleeve member in a body cavity according to some embodiments of the present application.

### DETAILED DESCRIPTION OF THE EMBODIMENTS

Improved apparatuses and methods are provided for handling post-operative complications. The apparatuses and methods disclosed herein are particularly useful for handling post-operative complications that result from abdominal surgeries, including gynecologic and urologic procedures. The apparatuses and methods described herein allow for a surgeon to immediately insufflate an abdomen of a patient post-operation to scan for and resolve post-operative complications. The apparatuses and methods described herein also help to reduce the risk of damage to internal organs post-operation.

More specifically, the improved apparatuses and methods relate to an improved “indwelling” trocar capable of being maintained in a patient for a certain period of time following a completed surgical procedure. The improved trocar described herein can be used regardless of whether the original operation is: 1) laparoscopic or 2) open. While conventional trocars may need to be re-inserted following a surgical procedure thereby leaving internal organs at particular risk, the improved indwelling trocar of the present application would allow safe and easy reexamination of intra-abdominal organs (e.g., by using existing 5 mm laparoscopic cameras) with minimal risk to internal organs.

In some embodiments, an improved trocar is provided that is kept in a patient following completion of a surgical procedure. The trocar comprises an “indwelling” trocar that remains in a patient for a certain period of time even after the patient's body is deflated. The indwelling trocar serves as an access path that can be used for immediate delivery of one or more cameras (e.g., 5 mm or smaller cameras) post-operation to scan and monitor for complications, as well as for immediate delivery of surgical instruments to resolve any post-operative complications. By allowing the indwelling trocar to remain in the patient for a certain period of time following a surgical procedure, rather than having to re-insert a trocar as is the conventional practice, the risk of injury to internal organs during post-operation procedures is reduced. Moreover, as the indwelling trocar is already in the patient, post-operation procedures can be performed much more quickly than conventional procedures, as there is no need to re-insert the trocar in the patient. In addition, with the indwelling trocar left behind in a patient, reopening an incision by re-insertion of a trocar will not be necessary unless a problem is found in a patient. In some embodiments, the indwelling trocar with the obturator removed allows reinflation of the abdomen and insertion of addition trocars under direct vision with the abdomen insufflated.

### Trocar Assemblies

In accordance with embodiments of the present application, an improved indwelling trocar is provided that comprises a cannula and an obturator. In some embodiments, the cannula includes a first section and a second section distinct from the first section. In some embodiments, the second section of the cannula remains in a patient's body post-operation for a certain period of time. The first section of the cannula can be comprised of a material such as polyetherether ketone (PEEK) that is unfilled, glass-filled or carbon-filled, glass-fiber, rigid plastics including polycarbonates, acrylates, polyvinylchlorides, polypropylene, polysulfone, polymethylmethacrylate, polystyrene, and copolymers, composite resins, or similarly rigid materials. The second section of the cannula can be comprised of a material that is softer and/or more flexible than that of the first section, including soft elastomers comprising natural rubber, synthetic polyisoprene, butyl rubber, polybutadiene, styrene-butadiene rubber, nitrole rubber, chloroprene rubber, ethylene propylene rubber, epichlorohydrin rubber, polyacrylic rubber, silicone rubber, fluorosilicone rubber, fluoroelastomers, perfluoroelastomers, polyether block amides, chlorosulfonated polyethylene, ethylene-vinyl acetate, polypropylenes, as well as soft silicone, synthetic latex, or an equivalent soft and/or flexible material. In some embodiments, the first section of the cannula is comprised of a hard plastic material, while the second section is comprised of silicone or rubber. One skilled in the art will appreciate that the cannula is not limited to those materials described above. In some embodiments, the first section of the cannula is comprised of a material having a modulus of elasticity greater than 3.5 GPa, while the second section of the cannula is comprised of a material having a modulus of elasticity less than or equal to 3.5 GPa. By having a second section that is comprised of a material that is softer and more flexible than the first, the cannula is capable of remaining in a patient post-operation for a certain period of time with minimal risk of injury to the patient's internal organs. This is in contrast to conventional trocar cannulas, which are generally comprised of a single rigid material and are not capable of being maintained in a patient post-surgery following deflation of the anatomical cavity due to the inadvertent risk of puncturing or laceration to internal organs by the rigid cannula body. In some embodiments, the first and second sections of the cannula form a continuous body with an axial bore formed therethrough, through which an obturator may be slideably inserted.

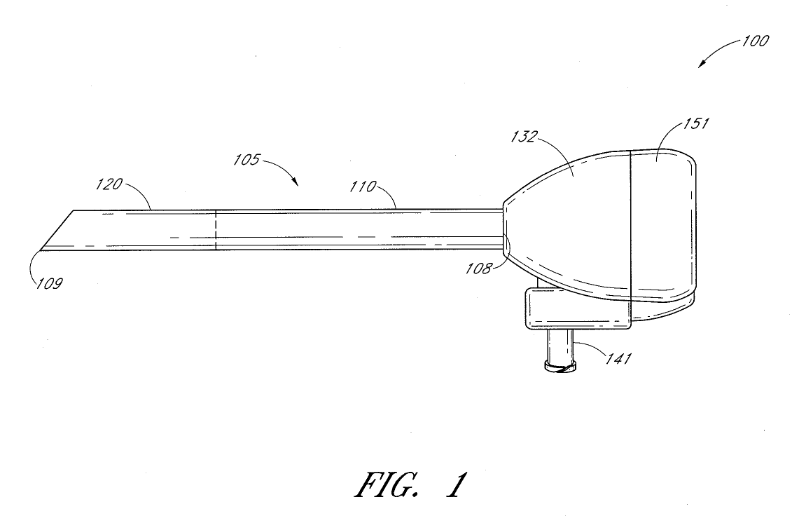

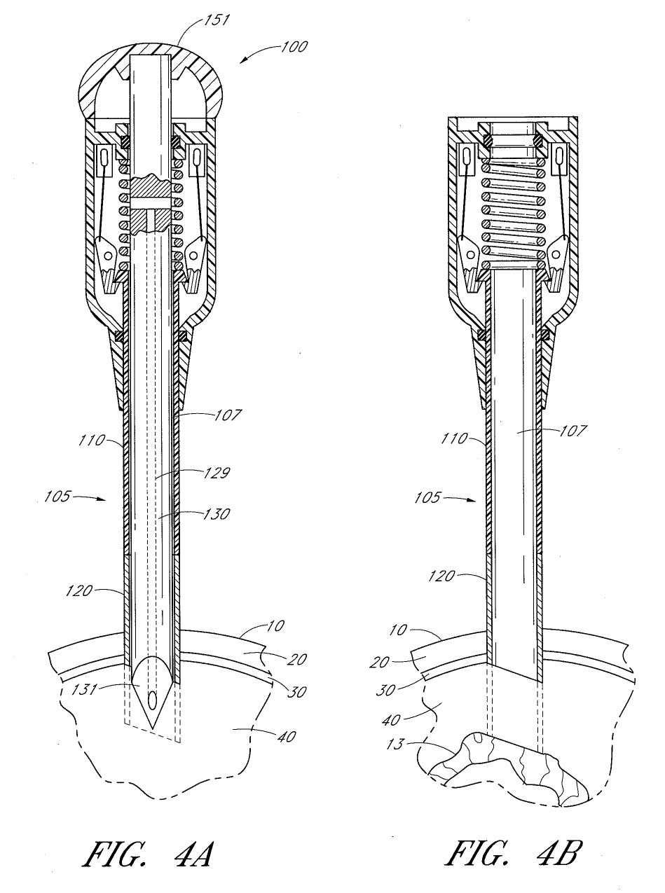

FIG. 1 illustrates a side view of a trocar 100 according to embodiments of the present application. The trocar 100 includes a cannula 105 attached at its proximal end 108 to a funnel-shaped fixture 132. The trocar cannula 105 also comprises an internal bore 107 (shown in FIGS. 4A and 4B) through which an obturator having a handle 151 can be slideably inserted.

As shown in FIG. 1, the trocar cannula 105 comprises a first section 110 and a second section 120. The cannula 105 includes an axial bore that runs through the first and second sections. The first section 110 includes the proximal end 108 of the cannula and is comprised of a first material. The second section 110 includes the distal end 109 of the cannula and is comprised of a second material. In some embodiments, the second section comprises a skirt or tube that is continuous with the first material. In a preferred embodiment, the second section 110 is of a material that is softer and/or more flexible than the first section. By being of a softer and/or more flexible material than the first section 110, the second section 120 can be placed proximate to and/or abut internal organs without damaging the internal organs. The trocar can thus be safely maintained in a patient both during surgery and for a certain period post-surgery if desired, even after the patient's body has been deflated. In some embodiments, using a trocar having a softer and/or more flexible second section 120 reduces the risk of damage to internal organs in a patient during surgery and/or post-surgery. The risk of damage to internal organs during surgery or post-surgery can be reduced by between 30% and 90%, and in some embodiments, by 95% or more.

In some embodiments, the first material of the first section 110 comprises a hard or substantially rigid material such as a hard or rigid plastic, while the second section 120 comprises a material softer and more flexible than the first material, including silicone, rubber, or other elastomer. In some embodiments, the first material of the first section 110 has a modulus of elasticity that is greater than 2 GPa, while the second material of the second section 120 has a modulus of elasticity that is less than 2 GPa.

As shown in FIG. 1, the first section 110 and the second section 120 of the trocar cannula 105 form a continuous cylindrical body. While the first section 110 and the second section 120 can comprise two separate pieces that are joined together (e.g., by molding) to form a continuous body, in some embodiments, the first section 110 and the second section 120 are formed from a single, monolithic piece. In some embodiments, the total length of the trocar cannula 105 comprising the first section 110 and second section 120 is between about 9 cm and 17 cm. The length of the first section 110 including the proximal end of the cannula is between about 6 cm and 14 cm in length. The length of the second section 120 including the distal end of the cannula is between about 3 cm and 7 cm in length, and more preferably about 3 cm, 5 cm, or 7 cm, depending on the size of the patient. However, other dimensions are possible. While in some embodiments, the wall-thickness of the first material of the first section 110 is about equal to the wall-thickness of the second material of the second section 120, in other embodiments, the wall-thickness of the first material can be slightly greater than the second material. In other embodiments, the wall-thickness of the second material can be slightly greater than the first material to provide sufficient mechanical strength to prevent collapsing of the second section 120. For example, in some embodiments, the second section 120 can comprise a tube-like material that has a slightly greater wall-thickness than the first section 110 to provide sufficient bending strength, resistance to buckling, and shear strength.

In some embodiments, the distal end 109 of the second section 120 can be flat or blunt, while in other embodiments (as shown in FIG. 1), the distal end 109 of the second section 120 can be beveled. Having a beveled distal end 109 allows for easier insertion of the cannula 105 into a patient.

The proximal end 108 of the first section 110 of the cannula 105 is connected to a funnel-shaped fixture 132. The funnel-shaped fixture 132 includes a valve 141, which can be used to provide an insufflating gas into the body of the patient during surgery or post-surgery. The funnel-shaped fixture 132 also includes a top opening (not shown), through which an obturator can be slideably inserted.

The obturator comprises a piercing element (e.g., one or more blades comprised of stainless steel or more preferably, bladeless piercing elements) capable of providing an incision through a patient's abdominal cavity. In some embodiments, the piercing element is at the distal end of an obturator shaft that is slideably inserted into the axial bore of the cannula 105. The obturator shaft is connected to a handle 151, the base of which is coupled to the top of the funnel-shaped fixture 132. The handle 151 of the obturator is operated by the surgeon, and allows the surgeon to control the piercing element of the obturator. In some embodiments, the obturator includes a shield member which assists in reducing the risk of damage to internal organs when the obturator pierces the abdominal cavity of the patient.

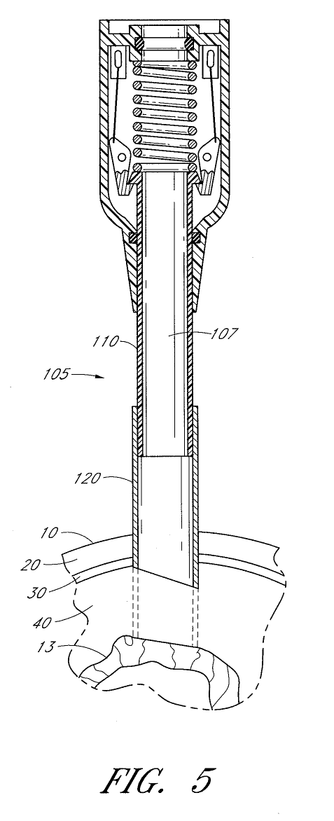

In other embodiments, an indwelling trocar can be provided having a first section 110 comprising a rigid material and a second section 120 comprising a sleeve member comprised of a substantially rigid, yet softer and/or more flexible material than that of the first section 110. The cross-section of an embodiment of an indwelling trocar having a sleeve member is illustrated in FIG. 5. While in some embodiments the sleeve member can be attached to the end of the first section 110 (e.g., by applying an adhesive), in other embodiments, the sleeve member can overlie and cover at least a portion of the first section 110 (as shown in FIG. 5). The materials of the first section 110 and the second section 120 comprised of the sleeve member are similar to those described above. For example, in some embodiments, the first section 110 can be comprised of a hard plastic material, while the second section 120 comprising a sleeve member can be comprised of silicone or rubber.

In yet other embodiments, an indwelling trocar can be provided having a cannula formed of a single material, rather than a first and second section comprised of different materials. In some embodiments, the single material chosen provides enough rigidity to the cannula to avoid soft tissue compression and collapse in the subcutaneous location, while also allowing for safe placement of the trocar cannula at or near internal organs, thereby allowing the trocar to be maintained in a patient for a certain amount of time following completion of a surgical procedure. For example, in some embodiments, the entire trocar cannula can be comprised of silicone or rubber with enough rigidity to avoid soft tissue compression. Like the trocar cannulas comprised of first and second sections, the indwelling trocar formed of a single material can be kept in a patient for a certain duration following completion of a surgical procedure.

### Methods

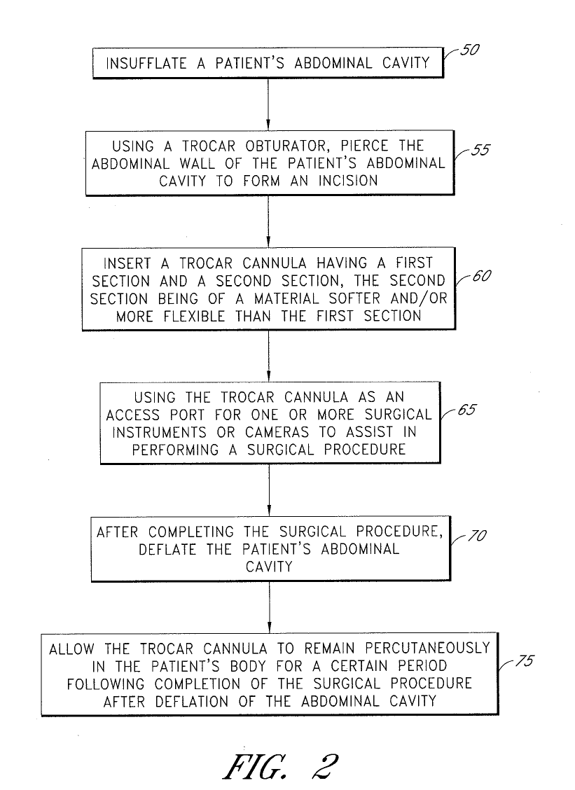

Methods are described using an improved trocar assembly in accordance with the descriptions above. FIG. 2 is a flow chart of a method of laparoscopic surgery using an improved trocar assembly according to some embodiments of the present application. The method comprises insufflating 50 a patient's anatomical cavity (e.g., abdominal cavity); piercing 55 the abdominal wall of the patient's abdominal cavity to form an incision by using an obturator after forming a small, initial skin incision; inserting 60 a trocar cannula having a first section and a second section percutaneously through the incision to form an access port for one or more surgical instruments or cameras; performing 65 a surgical procedure while using the trocar cannula as an access port for one or more surgical instruments or cameras; deflating 70 the patient's abdominal cavity after completing the surgical procedure; and allowing 75 the trocar cannula to remain percutaneously in the patient's body for a certain period following completion of the surgical procedure after deflation of the abdomen. In some embodiments, the indwelling trocar cannula can be removed at bedside and the skin incision closed (with steri strips or Dermanbond glue) when the indwelling trocar is no longer needed.

In accordance with the flow chart in FIG. 2, a patient's abdominal cavity is insufflated 50 with a non-toxic gas, such as carbon dioxide. Following insufflation 50 of the patient's abdominal cavity, the abdominal wall of the patient's abdominal cavity can be pierced 55 by a piercing element of an obturator after forming a small, initial (e.g., 5 mm) skin incision. In forming the incision with the obturator, the obturator slideably extends through and beyond the distal end of a trocar cannula having a first section and a second section. The size of the incision can vary from patient to patient, depending on the type of surgery to be performed.

Once the incision is formed 55, the obturator can be retracted, and the trocar cannula having the first section and second sections can be inserted 60 percutaneously into the patient to form an access port for one or more surgical instruments or cameras. The first section includes the proximal end of the cannula that remains outside of the patient and closer to the surgeon, while the second section includes the distal end of the cannula that is maintained within the abdominal cavity of the patient (as shown in FIG. 4B). The second section of the cannula is comprised of a material that is softer and/or more flexible than the material of the first section. In some embodiments, the softer and/or more flexible material of the second section of the cannula reduces the damage to internal organs during the surgical procedure, while in other embodiments, the softer and/or more flexible material of the second section helps to reduce the risk of damage to internal organs during surgery. The softer and/or more flexible material of the second section of the cannula also allows the cannula to be safely maintained in a patient for a certain duration following completion of a surgical procedure. The cannula forms an access port through which one or more surgical instruments or cameras can be delivered therethrough. Surgical instruments that can be delivered through the cannula include scissors, dissectors, graspers, retractors, probes, staples/cutters, and cameras (e.g., 5 mm cameras).

In accordance with the flow chart in FIG. 2, a surgical procedure can be performed 65 using the trocar cannula as an access port. In some embodiments, the surgical procedure can comprises numerous procedures and is not limited to the following: bariatric surgery including lap bands, Roux-En-Y gastric bypass, sleeve gastrectomy; laparoscopic Nissen fundoplication; laparoscopic gastric and intestinal resections; colonic resections; open ischemic bowel resections; colon resections; trauma surgery, and all intra-abdominal surgeries whether open or laparoscopic. This indwelling cannula would be particularly useful in very obese patients and critically ill patients, and in those patients who have underwent hazardous initial operations where a “second look” for post-operative complications is often mandatory. After completing one or more surgical procedures, the patient's abdominal cavity is deflated 70. The trocar cannula is allowed 75 to remain percutaneously in the patient's body for a certain period of time post-surgery following deflation of the abdominal cavity. In some embodiments, the trocar cannula can remain in the patient's body following a surgical procedure for a period between about one and seven days, more preferably between about one and two days. In some embodiments, the trocar cannula remains in the patient's body following a surgical procedure for less than a 24 hour period, e.g., for 12 hours or less. In some embodiments, the trocar assembly can be used in other procedures to provide access to other anatomical cavities, besides or in addition to the abdominal cavities, during a surgical procedure.

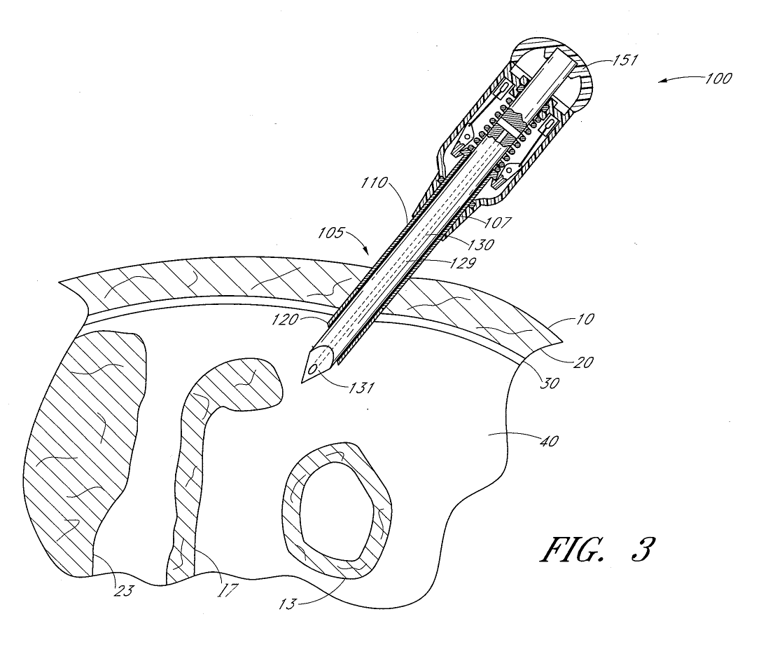

FIGS. 3-4B illustrate different stages of an indwelling trocar assembly in operation near a surgical site according to some embodiments. FIG. 3 illustrates a cross-sectional view of an improved trocar that has pierced through the abdominal wall of the patient's abdominal cavity. The trocar 100 comprises a cannula 105 having a first section 110 and a second section 120 made of a material softer and more flexible than the first section. The first and second section of the cannula form a continuous cylindrical piece with a bore 107 therethrough. The trocar 100 also comprises an obturator 129 having a shaft 130 and a piercing tip 131. The obturator 129 is slideably inserted into the bore 107 of the cannula 105.

As shown in FIG. 3, the piercing tip 131 of the obturator 129 can pierce the subcutaneous tissue and fascia 10, the pre-peritoneal space 20 and the peritoneum 30, until the distal end of the trocar cannula 100 is within the abdominal cavity 40 of the patient. The abdominal cavity 40 houses internal organs, including the transverse colon 13, the gall bladder 17 and the liver 23. In some embodiments, these internal organs can serve as surgical site locations. As shown in FIG. 3, during surgery, the distal end of the trocar 100 can be positioned near the internal organs. Preferably, measures are taken to prevent accidental piercing of the internal organs by the piercing tip 131 of the obturator 129 (e.g., by providing a shield or other protective mechanism). Once a small, initial skin incision is made with a scalpel, the trocar 100 can be inserted and, the obturator 129 can be slideably removed from the cannula bore 107, leaving the cannula 105 positioned percutaneously in the patient as an access port for surgical instruments.

FIG. 4A illustrates a cross-sectional view of a trocar 100 having a cannula 105 with a first section 110 and a second section 120 made of a material softer and more flexible than the first section and an obturator 129, while FIG. 4B illustrates the trocar 100 in FIG. 4A with the obturator 129 slideably removed from the cannula. When the obturator 129 is removed, the trocar cannula 105 remains in the patient's abdominal cavity 40. The trocar cannula can include a seal to prevent the leakage of insufflation gas from within the patient. Only a portion of the trocar cannula 105 remains in the patient's abdominal cavity 40 during the surgical procedure, as shown in FIG. 4B. The portion of the trocar cannula 105 that remains in the patient's abdominal cavity 40 generally comprises the second section 120 of the trocar cannula 105, which is comprised of a material that is softer and more flexible than the first section 110. As shown in FIG. 4B, by having a second section 120 comprised of a material softer and more flexible than the first section 110, the trocar cannula 105 can be located very close to internal organs, such as the bowel (e.g., the colon 13), with minimal risk of damage to the internal organs during surgery or post-surgery. In some embodiments, the distal end of the cannula 105 can safely abut one or more internal organs with minimal risk of damage to the internal organs during surgery or post-surgery.

FIG. 5 illustrates a cross-sectional view of an alternate trocar having a second section 120 comprised of a sleeve member in a body cavity according to some embodiments of the present application. Like the trocar illustrated in FIGS. 4A and 4B, the trocar having the sleeve member can be maintained in a patient during and after a surgical procedure. The sleeve member overlies the first section 110 of the cannula 105 at its distal end and can be kept very close to internal organs, such as the bowel, with minimal risk of damage to the internal organs during surgery or post-surgery.

In addition to the laparoscopic surgical procedures discussed above, the improved indwelling trocar of the present application can also be used in open surgeries. In some embodiments, an open surgical procedure comprises creating a first incision in a patient. This incision is generally much larger than the incisions used during laparoscopic surgeries. Following creation of the first incision, a surgeon can perform one or more surgical procedures through the first incision. At any time while the first incision is open, a trocar having an obturator and a cannula can be inserted through the first incision or via a separate small skin incision under the surgeon's control. Once the trocar is inserted, the first incision can be closed in part or completely and the obturator removed, while allowing the trocar cannula to be maintained for a desired period of time (e.g., between one and seven days) following closure of the first incision. In some embodiments, the trocar cannula is maintained in place following closure of the first incision, while in other embodiments, the trocar cannula can be shifted or angulated to various surgical locations once the first incision is closed. By maintaining the trocar cannula in the patient post-operation and after partial or complete closure of the first incision, the trocar cannula can serve as an access port for cameras and other equipment to check for post-operative complications.

In addition to the examples described above, one skilled in the art will appreciate that more than one indwelling trocar can be used in any operation. For example, it is possible to maintain one, two, three, four, five, six, seven, or more trocars in a patient following completion of a surgical procedure. In some embodiments, a combination of indwelling and non-indwelling trocars can be used.

It will be apparent to those skilled in the art that various modifications and variations can be made in the present invention without departing from the scope or spirit of the invention. Moreover, the indwelling trocar and method of use need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed indwelling trocar. Thus, it is intended that the present invention cover the modifications and variations of this invention provided that they come within the scope of the appended claims or their equivalents.

What is claimed is:

1. A method for performing a surgical procedure on a subject, comprising:
inflating an abdominal cavity;
inserting a trocar percutaneously to define an access path through the inflated abdominal cavity;
providing one or more instruments through the trocar to assist in performing a surgical procedure on the subject;
deflating the abdominal cavity following the surgical procedure; and
maintaining the trocar in the subject percutaneously for a desired period of time following deflation of the abdominal cavity.

2. The method of claim 1, wherein the trocar is maintained in the subject for up to three days.

3. The method of claim 1, wherein the trocar is maintained in the subject for up to seven days.

4. The method of claim 1, wherein inserting the trocar percutaneously to define an access path comprises:
introducing a trocar having a cannula and an obturator having a piercing end, wherein the obturator is inserted through a bore in the cannula;
piercing an abdominal wall of the inflated abdominal cavity with the piercing end of the obturator;
inserting the trocar having the cannula and the obturator percutaneously through the subject; and
removing the obturator from the cannula while maintaining the cannula in a percutaneous position to provide an access path.

5. A method for performing a surgical procedure in a body cavity of a patient, comprising:
deflating the body cavity following completion of a surgical procedure in the body cavity, wherein the surgical procedure is performed at least partially through a trocar cannula extending percutaneously through the patient's skin toward a surgical site; and
maintaining the trocar cannula percutaneously for a desired period of time following deflation of the body cavity, wherein the trocar cannula is configured to inhibit injury to tissue in the body cavity after deflation of the body cavity.

6. The method of claim 5, wherein the trocar is maintained percutaneously in a patient for between one and seven days following deflation of the body cavity.

7. The method of claim 5, wherein the trocar is maintained percutaneously in the same position for between one and seven days following deflation of the body cavity.

8. A method for performing an open surgical procedure in a body cavity of a patient, comprising:
creating an incision in the patient;
performing a surgical procedure through the incision;
inserting a trocar cannula percutaneously into a body cavity of the patient; and
following completion of the surgical procedure, closing at least a portion of the incision while allowing the trocar cannula to be maintained in place for a desired period of time after closure of the incision, thereby facilitating access to the body cavity of the patient through the trocar cannula after closure of the incision.

9. The method of claim 8, wherein the trocar cannula is inserted through the incision.

10. The method of claim 8, wherein the trocar cannula is inserted through a second incision separate from the incision in the patient.

11. A trocar, comprising:
a cannula having a first section, a second section and an axial bore, the second section comprised of a more flexible material than the first, the first section transitioning into the second section, the second section being located at the distal end of the trocar cannula, and wherein the axial bore runs through the first section and the second section; and
an obturator having a piercing end insertable into the cannula through the axial bore.

12. The trocar of claim 11, wherein the second section comprises a soft tube member.

13. The trocar of claim 12, wherein the soft tube member is comprised of silicon.

14. The trocar of claim 12, wherein the soft tube member is comprised of rubber.

15. The trocar of claim 11, wherein the first section is comprised of polypropylene and the second section is comprised of silicone or rubber.

16. The trocar of claim 11, wherein the second section is about 5 cm in length.

17. The trocar of claim 11, wherein the second section comprises a sleeve member that overlies at least a portion of the first section.

18. The trocar of claim 11, wherein the second section comprises a sleeve member that is attached to an end of the first section.

19. A trocar capable of remaining in a subject post-operation for a specified duration, comprising:
a cannula comprising a proximal end and a distal end, the cannula having a continuous body between the proximal end and distal end, wherein the distal end is comprised of a more flexible material than the proximal end, and wherein the flexible material comprises between 3 cm and 7 cm of the cannula body;
an axial bore formed through the cannula body; and
an obturator having a piercing end insertable into the cannula through the axial bore.

20. The trocar of claim 19, wherein the distal end is beveled.

21. The trocar of claim 19, wherein the distal end of the cannula comprises a separable part that is molded to the proximal end of the cannula to form the continuous body of the cannula.

22. The trocar of claim 19, wherein the distal end of the cannula and the proximal end of the cannula are formed from a monolithic piece.
